(19) United States
(12) Patent Application Publication
(10) Pub. No.: US 2026/0120796 A1
(43) Pub. Date: Apr. 30, 2026

(54) SIMULATED WHOLE EXOME SEQUENCING AND RNA SEQUENCING DATA FOR TUMOR CLONALITY

(71) Applicant: NEC Laboratories Europe GmbH, Heidelberg (DE)

(72) Inventors: Anja MOESCH, Heidelberg (DE); lsraa ALQASSEM, Heidelberg (DE)

(21) Appl. No.: 19/158,458

(22) PCT Filed: Nov. 3, 2023
(86) PCT No.: PCT/IB2023/061102

§ 371 Date: Aug. 21, 2025

Related U.S. Application Data
(60) Provisional application No. 63/533,366, filed on Aug. 18, 2023.

(51) Int. Cl. G16B 20/20; G16B 30/00

(52) U.S. Cl. CPC G16B 20/20; G16B 30/00

(57) ABSTRACT

A computer-implemented, machine learning method for generating clone-specific tumor data includes obtaining a phased transcriptome file, a phased transcript file, and a clonal structure that represents a tumor clonal structure and comprises one or more nodes. For each node of the clonal structure: input mutational pools are determined for mutating the phase transcriptome file and the phased transcript file; DNA sequence reads are sampled from the phased transcript file and the sampled sequence DNA reads are mutated; RNA sequence reads are sampled from the phased transcriptome file and the sampled RNA sequence reads are mutated; a mutated genome is generated using the mutated DNA sequence reads; and a mutated transcriptome is generated using the mutated RNA sequence reads. The method has applications including, but not limited to, use cases in medical AI/healthcare for optimization of predictions or to support decision-making.

### CROSS-REFERENCE TO PRIOR APPLICATION

This application is a U.S. National Phase application under 35 U.S.C. § 371 of International Application No. PCT/IB2023/061102, filed on Nov. 3, 2023, and claims benefit to U.S. Provisional Application Ser. No. 63/533,366 filed on Aug. 18, 2023, the entire contents of which is hereby incorporated by reference herein. The International Application was published in English on Feb. 27, 2025 as WO 2025/040949 A1 under PCT Article 21(2).

### FIELD

The present invention relates to Artificial Intelligence (AI) and machine learning (ML), in particular medical AI, and in particular to a method, system, computer program product, data structures containing models and/or generated data and computer-readable medium for generating tumor data.

### BACKGROUND

In most cases, tumor development starts with a single founder clone, i.e., a set of genetically identical cells. This clone arises from a single cell which undergoes genetic alterations or mutations, leading to uncontrolled cell division and therefore the formation of a tumor. As the tumor grows, daughter cells of the founder clone cells acquire different mutations or alterations leading to the development of additional subclones within the tumor mass, which can by described by a clonal (or phylogenetic) tree connecting subclones over time with the founder clone as its root.

The emergence of various subclones contributes to tumor's progression and heterogeneity (see Ewing, Adam D., et al., “Combining tumor genome simulation with crowdsourcing to benchmark somatic single-nucleotide-variant detection,” Nature Methods 12.7: 623-630 (2015), hereinafter “Ewing et al.”, which is hereby incorporated by reference herein). Typically, the investigation of tumor heterogeneity and clonal evolution is limited to the genomic level (or DNA level) mutations, specifically to single nucleotide variants and it mostly overlooks more complex variants like insertion or deletion of nucleotides in the DNA sequence or frameshift variants disrupting the reading frame during translation, which may lead to a completely different amino acid sequence and impact protein structure and function. Additionally, there are several other abnormal processes that occur at the transcriptomic level (RNA level) subsequent to DNA transcription. These processes involve RNA splicing followed by the translation of RNA into proteins. During RNA splicing, numerous tumor-specific events related to alternative splicing and gene fusion emerge which impact the final protein products. Therefore, when studying tumor clonality, providing for additional exploration of tumor-specific aberrant RNA events could allow to uncover additional tumor biomarkers and valuable immunotherapy vaccine targets since the investigation of the combined impact of RNA alterations and genomic mutations (including more complex variants like frameshifts) is greater than the impact of isolated genetic single point changes at the genomic level alone (see Salcedo, Adriana, et al., “A community effort to create standards for evaluating tumor subclonal reconstruction,” Nature Biotechnology 38.1: 97-107 (2020), hereinafter “Salcedo et al.”, which is hereby incorporated by reference herein).

### SUMMARY

In an embodiment, the present invention provides a computer-implemented, machine learning method for generating clone-specific tumor data using artificial intelligence (AI). A phased transcriptome file, a phased transcript file, and a clonal structure that represents a tumor clonal structure and comprises one or more nodes is obtained. For each node of the clonal structure: input mutational pools are determined for mutating the phase transcriptome file and the phased transcript file; DNA sequence reads are sampled from the phased transcript file and the sampled sequence DNA reads are mutated; RNA sequence reads are sampled from the phased transcriptome file and the sampled RNA sequence reads are mutated; a mutated genome is generated using the mutated DNA sequence reads; and a mutated transcriptome is generated using the mutated RNA sequence reads. The method has applications including, but not limited to, use cases in medical AI/healthcare for optimization of predictions or to support decision-making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a method and system according to an embodiment of the present invention for simulating DNA (genomic) and RNA (transcriptome) data;

FIG. 2 is a flow diagram of a method to generate tumor data according to an embodiment of the present invention; and

FIG. 3 is a block diagram of an exemplary processing system, which can be configured to perform any and all operations disclosed herein;

### DETAILED DESCRIPTION

Embodiments of the present invention provide a system and methodology for generating simulated tumor clonality datasets (whole exome sequencing (WES) and RNA sequencing (RNA-seq)) based on real-world observations from cancer samples. The approach considers a tumor evolution mechanism and the combined impact of genomic mutations (DNA-level) and transcriptomic alterations (RNA-level). This is in contrast to existing approaches which are limited to simulating tumor clonality based on DNA-level mutations alone. Accordingly, embodiments of the present invention provide for improvements to computer functionality in an AI system, in particular enhancing the computer functionality to consider the combined impact of genomic mutations (DNA-level) and transcriptomic alterations (RNA-level) and improving the accuracy and performance of the AI system with the simulated tumor clonality datasets. This improved accuracy and performance supports decision making and optimization of predictions by the AI system and, in particular, provides for further improvements in AI assisted drug and vaccine design, for example, by providing for improved predictions of tumor behavior, guiding therapeutic target identification in immunotherapy-based vaccine, and aiding in the evaluation of various treatment strategies, ultimately leading to improved patient outcomes.

As mentioned above, tumor development typically begins with a single founder clone, derived from a cell that undergoes genetic mutations or transcriptomic alterations, causing uncontrolled cell division and tumor formation. Such abnormalities in the founder clone provide a growth advantage over healthy cells. As the tumor evolves, daughter cells acquire further changes, creating additional subclones within the tumor. Being able to more accurately predict and simulate tumor clonal structure not only contributes to the understanding of cancer biology, but also assists in predicting tumor behavior, allowing to guide therapeutic target identification in immunotherapy-based vaccine, and aiding in the evaluation of various treatment strategies, ultimately leading to improved patient outcomes. Existing approaches which simulate tumor progression and heterogeneity are limited to simulating genomic-level mutations and overlook transcriptomic-level alterations. In contrast, embodiments of the present invention provide a system and method for generating simulated tumor clonality datasets, based on real-world data, and considering tumor evolution and the combined impact of both genomic mutations and transcriptomic alterations.

Being able to cover all subclones by incorporating at least one immunogenic event (e.g., genomic mutation or RNA alteration event) of each subclone into the vaccine formula would allow to create a more efficient cancer immunotherapy-based vaccine or treatment.

Ideally, DNA mutations and RNA alterations of the founder clone would be leveraged for this purpose, however they are not always identifiable and might not be immunogenic, and therefore cannot be targeted by vaccine elements. Thus, this presents the technical problem of how to know which DNA mutation and/or RNA alteration belongs to which subclone. To solve this technical problem, methods need to be developed using data for which the exact composition of subclones and their mutations are known. There are two types of data that could be used to solve the problem. First, single cell sequencing can be used as an experimental method to produce information about each individual cell including its genomic mutations and transcriptomic alterations. However, identification of mutations in single cells is difficult (see Robinson, Mark D., et al., “edgeR: a Bioconductor package for differential expression analysis of digital gene expression data,” Bioinformatics 26.1: 139-140 (2010), hereinafter “Robinson et al.”, which is hereby incorporated by reference herein) and single cell sequencing itself is very costly. Second, bulk sequencing data could be used. This is relatively cheap and the current standard approach of acquiring genetic information from patients uses bulk sequencing data. However, since there is no experimental method to determine the ground truth clonal structure in bulk sequencing samples, a method based on this type of sequencing data cannot be as accurate as single cell sequencing if no additional information is provided (e.g., sequencing multiple samples from different regions of the tumor or sequencing the tumor mass at different time points). However, analyzing multiple samples from a single patient is a costly process that imposes an additional burden on the patient and may not be feasible in some cases.

Since only a very limited amount of ground truth data is available, simulated or synthetic data can be used to develop computational methods to identify subclones. This data includes sequencing data (Binary Alignment/Map files referred to as BAM files) from a healthy individual that is modified (mutated) according to a desired clonal structure (e.g., the tree of clonal evolution).

Existing methods and pipelines for simulating data of tumor clonal evolution are focused on DNA and try to cover all aspects of tumor mutation equally regardless of the density (or abundance) of each subclone within the tumor mass. However, for immunotherapy vaccine development, embodiments of the present invention recognize that certain aspects are more important (e.g., the association of all mutations to identified subclones compared to the clonal tree structure). Additionally, embodiments of the present invention recognize that more complex tumor-specific variants like frameshifts, gene fusions and alternative splicing events are of high interest since they represent the most promising vaccine targets. In contrast to embodiments of the present invention, existing technology is not able to cover these important aspects or complex tumor-specific variants at all.

Obtaining real-world tumor evolution data with known ground-truth labels which assign DNA mutation and RNA variants to different clones at time points can be expensive and even impossible, thus simulated data provides an alternative, efficient approach to overcome the technical problem of limited data. Embodiments of the present invention provide a method and system to generate synthetic tumor clonal data by combining DNA and RNA variants. The approach according to embodiments of the present invention ensures reliable labels by deliberately assigning distinct DNA mutations and RNA variants to each clone, considering mutations and variants at the parent clones. Although tumor evolution is complex and not fully understood, the simulated data paves the way towards developing and benchmarking more accurate tumor clonality approaches, which allows to more accurately predict and prioritize neoantigen targets that cover all tumor clones when developing immunotherapy-based vaccines. Furthermore, the ability to inexpensively and quickly generate large amounts of synthetic data with known ground truth supports the development of machine learning based approaches for deciphering tumor clonality.

Embodiments of the present invention enhance the computer functionality of AI systems to generate synthetic tumor sequencing data that resemble real-world dataset, which can be used to increase the accuracy and performance of AI tools, for example, being used to predict targets for vaccine development.

In a first aspect, the present invention provides a computer-implemented, machine learning method for generating clone-specific tumor data. A phased transcriptome file, a phased transcript file, and a clonal structure that represents a tumor clonal structure and comprises one or more nodes is obtained. For each node of the clonal structure: input mutational pools are determined for mutating the phase transcriptome file and the phased transcript file; DNA sequence reads are sampled from the phased transcript file and the sampled sequence DNA reads are mutated; RNA sequence reads are sampled from the phased transcriptome file and the sampled RNA sequence reads are mutated; a mutated genome is generated using the mutated DNA sequence reads; and a mutated transcriptome is generated using the mutated RNA sequence reads.

In a second aspect, the present invention provides the method according to the first aspect, further comprising modifying the mutated genome or the mutated transcriptome by inserting random alterations.

In a third aspect, the present invention provides the method according to the first or the second aspect, further comprising modifying the mutated genome or the mutated transcriptome by removing one or more nodes of the clonal structure.

In a fourth aspect, the present invention provides the method according to any of the first to third aspects, wherein determining the input mutational pools for mutating the phased transcriptome file and the phased transcript file includes restricting a pool of mutations using observed cancer-type specific mutational signatures associated with the clonal structure.

In a fifth aspect, the present invention provides the method according to any of the first to fourth aspects, wherein determining the input mutational pools for mutating the phased transcriptome file and the phased transcript file further includes restricting the pool of mutations using observed cancer-type specific alternative splicing and gene fusion events, and wherein determining the input mutational pools and restricting the pool of mutations includes updates from database.

In a sixth aspect, the present invention provides the method according to any of the first to fifth aspects, wherein the phased transcriptome file and the phased transcript file are Binary Alignment/Map (BAM) files.

In a seventh aspect, the present invention provides the method according to any of the first to sixth aspects, wherein generating the mutated genome using the mutated DNA sequence reads includes merging sequence reads simulated from the mutated DNA sequence reads of each node of the clonal structure.

In an eighth aspect, the present invention provides the method according to any of the first to seventh aspects, wherein merging the sequence reads simulated from the mutated DNA sequence reads further includes using parameters identifying sequence error rates and down sampling from each node of the clonal structure.

In a ninth aspect, the present invention provides the method according to any of the first to eighth aspects, wherein sampling the RNA sequence reads includes using a negative binomial distribution.

In a tenth aspect, the present invention provides the method according to any of the first to ninth aspects, wherein mutating the sampled RNA sequence reads includes adding DNA variants.

In an eleventh aspect, the present invention provides the method according to any of the first to tenth aspects, wherein mutating the sampled RNA sequence reads includes augmenting with genomic mutations specific to a node of the clonal structure that corresponds to the DNA at the same position as the RNA for the node.

In a twelfth aspect, the present invention provides the method according to any of the first to eleventh aspects, further comprising sorting sequence reads by coordinates of the mutated DNA sequence reads and the mutated RNA sequence reads.

In a thirteenth aspect, the present invention provides the method according to any of the first to twelfth aspects, wherein sampling the DNA sequence reads and the RNA sequence reads includes using sliding window sampling.

In a fourteenth aspect, the present invention provides a computer system for generating tumor data comprising one or more processors, which, alone or in combination, are configured to perform a machine learning method for generating tumor data according to any of the first to thirteenth aspects.

In a fifteenth aspect, the present invention provides a tangible, non-transitory computer-readable medium for generating tumor data which, upon being executed by one or more hardware processors, provide for execution of a machine learning method according to any of the first to thirteenth aspects.

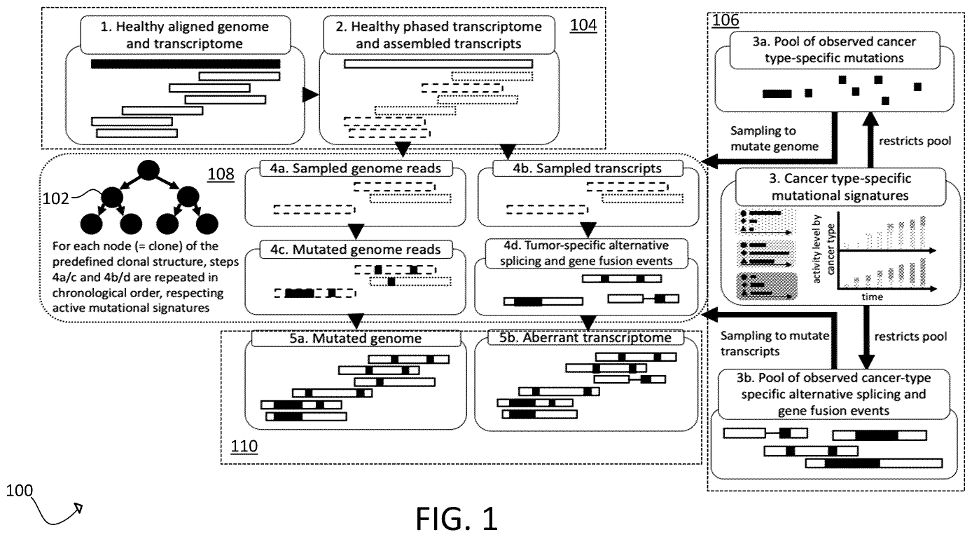

The system according to an embodiment of the present invention takes as input two BAM files, one contains aligned DNA sequencing reads from a healthy individual and the other contains aligned RNA sequencing reads from the same individual (see FIG. 1, Step 1). These files represent the baseline or reference dataset that will be used as the starting point before introducing DNA mutations and RNA variants. Further, the system takes three additional files as input. One file contains cancer type-specific mutational signatures and their associated cancer types and timepoints of activeness (see FIG. 1, Step 3). This file can contain information about which mutation can be inserted at which time points (i.e., when the mutational signature is active). For example, UV-induced mutational signatures are active in early-stage melanoma. The second file is a pool of observed (e.g., from cancer patients) cancer mutations, which includes information for each individual mutation, such as the type of mutation (e.g., single-nucleotide variants, insertions, deletions, frameshift and also larger structural variants like chromosomal duplications), their genomic coordinates, and the associated mutational signatures if this information is available (see FIG. 1, Step 3a). The third file contains cancer type-specific alternative splicing and gene fusion events, also linked to mutational signatures if possible (see FIG. 1, Step 3b). The latter two files cover the genomic mutations and transcriptomic variants to be introduced into the BAM files. These files containing information about mutational signatures and their associated mutations/transcript variants observed in cancer patients can be regularly updated by accessing publicly available databases, like the Catalogue Of Somatic Mutations In Cancer (COSMIC) database, to ensure that the pool of mutations and transcript variants reflects the state-of-the-art knowledge about cancer aberrations.

FIG. 1 illustrates a workflow 100 of a system according to an embodiment of the present invention for simulating DNA (genomic) and RNA (transcriptome) according to a tumor clonality tree. Starting at Step 1, from healthy genome/transcriptome data, reads are sampled and mutated for every tumor clone following the timeline of clonal evolution and taking mutations/RNA variants according to the active mutational signature into account in workflow 100. In particular, the system according to an embodiment of the present invention starts from a predefined clonality (phylogenetic) tree which resembles tumor clonal structure (a tree with multiple child nodes, as seen at 102), where each node can have a single or multiple child nodes. The structure of the tree itself is not limited (e.g., a parent node can have one or multiple children and not all branches need to have the same depth). FIG. 1 shows a simplified example at 102. The depth of the tree resembles how the tumor evolves over time. The clonal tree depth and the fraction (abundance or ratios) of reads at each node can be parametrized based on real-world ground truth tumor data, if available. The clonal tree depth and the fraction of reads at each node can be provided by a user. The first node is referred to as the founder clone and it uses the healthy DNA and RNA sequencing reads as inputs. For the sequencing reads, the system applies an existing phasing algorithm to determine which reads originate from which parent chromosome (share mutations when duplicated) by using tools like ProbHap or HapCUT2 (see FIG. 1, Step 2). Phasing may refer to a process for generating separate sequences which represent a variant arrangement on chromosomes that allows the ability to identify which variants are inherited together. This advantageously provides to ensure that mutations occurring on the same chromosomal copy are inherited together to the following subclones and correctly multiplied by larger structural variants like whole chromosome copy events. In embodiments, Step 1 and Step 2 may be executed by a data preparation module 104.

For DNA simulation via workflow 100, following the predefined clonal structure, the steps below are repeated for each clone using the parent clone DNA reads as input (see also FIG. 1, Steps 4a and 4c). A subset of reads is sampled from the input taking the fraction of each clone into account. The sliding window sampling approach described by (see Salcedo et al.) can be applied to ensure that the whole genome is covered. Then, mutations are inserted. The mutations are sampled from a distribution that reflects the expected distribution of mutations in the population of interest, in particular a pool of mutations that is restricted by the cancer type and the currently active mutational signatures which differ from one time point to another, in particular in the depth in the clonal tree (see Steps 3, 3a, and 3b). Steps 3, 3a, and 3b may be executed by mutation pool generation module 106.

After that, the reads simulated for each clone (node) are merged considering various parameters, such as (i) sequencing error rates to generate reads that resemble those obtained from a real sequencing experiment, and (ii) if necessary, down sampling of reads in each node to ensure uniform sequencing depth across various clones (see FIG. 1, Step 5a).

In an embodiment, an RNA simulation via workflow 100 is provided which begins with aligned RNA sequencing (RNA-seq) reads contained in a BAM file from a healthy individual. To assemble transcripts and estimate their abundance, established tools like StringTie can be utilized. The founder clone contains RNA-seq reads simulated based on the RNA-seq reads of the healthy individual. Subsequently, for each clone, an embodiment of the present invention can be used to simulate tumor-specific alternative splicing events using the following steps (see also FIG. 1, Steps 4b and 4d):
1. The process is initiated using the parent clone's RNA-seq reads. A subset of reads is sampled from the input ensuring that the full genome is covered and taking the fraction of each clone into account. The negative binomial distribution is used for RNA transcript sampling, since it has been demonstrated to effectively capture both biological and technical variability when used to model read counts (see Robinson et al. and Frazee, Alyssa C., et al., “Polyester: simulating RNA-seq datasets with differential transcript expression,” Bioinformatics 31.17: 2778-2784 (2015), which is hereby incorporated by reference herein). Although a negative binomial distribution is described as an example, other count data models can be used for RNA transcript sampling.
2. Cancer-specific events are sampled from a pool of cancer type-specific alternative splicing and gene fusion events. Then, all the RNA-seq reads are retrieved that cover the selected events. These reads replace the ones from the parent node which overlap the same genomic locations.
3. RNA-seq reads are incorporated that align with genomic mutations specific to that clone as outlined in the DNA simulation workflow. Additionally, the mutations present in the DNA at the same position in the RNA are augmented. Mutations present in the DNA are copied to the RNA to be present at the same position. This inclusion is particularly advantageous for improving performance since certain mutations, such as splicing-induced mutations, can impact splicing mechanism.
4. To generate a new set of RNA-seq data for each clone, transcript assembly and abundances are estimated. These values serve as inputs for an RNA-seq simulator, such as Polyester or RNA-Seq by Expectation-Maximization (RSEM). Steps 4a, 4c, 4b, and 4d may be performed by sampling and mutating module 108.

As for DNA, the RNA reads are then merged to one file containing the aberrant transcriptome (see FIG. 1, Step 5b). This file can also be a BAM file. Merging the mutated genome and aberrant transcriptome (e.g., Steps 5a and 5b) may be executed by merging module 110 and with the use of a merge function provided by SAMtools software package.

The number of mutations and splicing variations introduced at each step can be defined by the mutational rate of the simulated cancer type and should typically vary dependent on the time point. In embodiments, the number of mutations and splicing variations can be provided as input or by using the cancer type as an input parameter.

Finally, random noise can be added to the simulated clonal tree, such as by removing clones or adding random mutation events to resemble empirical data. The removal of clones or adding random mutation events can also be based on user specifications. By completing DNA and RNA simulation workflows, each clone will encompass genomic mutations, alternative splicing, and gene fusion events. The reads of individual clones will be merged and sorted by coordinate using a utility such as SAMtools to make it impossible to identify which read belongs to which clone by order of reads thereby avoiding the need to use the read position by software that analyzes the data.

Embodiments of the present invention can be applied, for example, to the field of digital medicine to improve the accuracy and performance of AI systems, such as those used for drug or vaccine development. Two exemplary use cases for which the incorporation of the simulation of synthetic tumor data with underlying clonal information can be incorporated into an AI tool (the data is meant to close the gap for developing methods predicting clonality including number of clusters (or clones) and their compositions (mutations, RNA splicing, gene fusions)) are:
1. Synthetic tumor clonality data can be used to evaluate methods based on probabilistic modeling, in particular methods that use prior biological knowledge to create inference models.
2. Synthetic tumor clonality data can be used to train and evaluate supervised machine learning/deep learning models, e.g., clustering methods. This is a new concept that has not been explored yet due to the lack of suitable data and ground-truth labels and the limitations of existing technology. The technology provided according to embodiments of the present invention (also referred to as “OncoCloneSim”) provides the assigned cluster labels (or clone) for each group of mutation, and provides to simulate enough tumor samples with a sufficient quality to make exploring machine learning approaches feasible.

In an embodiment, the present invention provides a method for simulating clone-specific tumor DNA and RNA, the method comprising the steps of:
1) Providing input in the form of matched healthy DNA and RNA aligned BAM files and desired clonal structure (see FIG. 1, Step 1). The input can be provided as input or defined based on different pipelines which are used depending on the type of cancer.
2) Phasing of BAM files (see FIG. 1, Step 2).
3) Creating input mutational pools, signatures and observed cancer specific alternative splicing (see FIG. 1, Steps 3, 3a and 3b).
   a. Advantageously, the creation of both pools and mutational signatures could also be updated in an automated fashion, using publicly available and regularly updated databases like COSMIC.
4) For each clone:
   a. Sampling and mutating DNA reads (+keeping mutations of previous parental clones).
   b. Sampling and mutating RNA reads (+adding DNA variants to transcripts). (see FIG. 1, Steps 4a and 4b)
5) Assembly of full genome and transcriptome (see FIG. 1, Steps 5a and 5b).
6) Adding noise (e.g., random alterations, removing clones).
7) Sorting reads by coordinates to avoid association of read order to clones (in some embodiments).

Embodiments of the present invention provide for the following improvements and technical advantages over existing technology:
1. Using tumor-specific mutations observed in cancer patients based on active mutational signatures to simulate tumor mutation. The set of mutational signatures restricts the pool for sampling mutations for each clone. Mutations can be, but are not limited to, single nucleotide variants, insertions, deletions, frameshifts, splicing-induced mutations.
2. To enhance understanding of tumor evolution, implementing parallel workflows that integrate both DNA and RNA data. This approach allows to consider the effects of transcriptomic aberrations alongside genomic mutations. Furthermore, it is ensured that genomic mutations are included in RNA reads. Tumor-specific alternative splicing and gene fusions serve as examples of transcriptomic aberrations. Embodiments of the present invention are not limited to these events only and can advantageously generalize to any additional types of transcriptomic aberrations observed in tumor tissues.
3. In contrast to existing technology, which is limited to primarily focus on single nucleotide variants occurring at the DNA level (overlooking, e.g., frameshifts and RNA aberrations), embodiments of the present invention enable to comprehensively include abnormalities at both the DNA and RNA levels, ensuring that the sequencing data from both DNA and RNA can be interdependent. Furthermore, the simulated data provided according to embodiments of the present invention is closer to real-world datasets since genomic and transcriptomic mutations that have been confirmed in cancer patients are used. Additionally, embodiments of the present invention incorporate noise and down sampling techniques to mimic real-world tumor samples, which often suffer from missing data and incomplete coverage of the entire tumor clonal structure.

BAMSurgeon (see Ewing et al.) is a system for simulating and introducing somatic mutations into real Next Generation Sequencing (NGS). BAMSurgeon can incorporate mutations into any alignment that is stored in BAM format. This includes RNA-seq (sequencing of transcriptome) and exome data (sequencing of protein-coding regions of the genome). The BAMSurgeon system implements a method that involves several steps. First, it identifies potential somatic mutations in a real tumor samples. It then uses this information to generate synthetic mutations. After that, the BAMSurgeon method selects genomic locations from an original BAM file which belongs to a normal tissue. The selection of these genomic locations is based on coverage information. Then, mutations are introduced by modifying reads that cover the selected genomic locations. Finally, the modified reads are merged back into the original BAM file. The synthetic BAM file together with the original BAM file represents tumor-normal pair which can be used in downstream analyses such as benchmarking somatic variant detection algorithms.

Embodiments of the present invention (OncoCloneSim) provide for a number of improvements over the BAMSurgeon technology, for example: (i) OncoCloneSim more accurately and effectively simulates tumor sequencing data taking tumor evolution and clonality into account, (ii) OncoCloneSim includes splicing variants and gene fusions to further improve accuracy, and (iii) instead of using synthetic mutations, OncoCloneSim samples tumor-specific DNA mutations and RNA variants directly from a pool of tumor samples, which offers the advantage of analyzing actual neoantigens that are observed in real tumor samples, thereby providing more realistic and relevant data for further analysis.

BAMSurgeon has been further developed and extended by Salcedo et al. in a pipeline aiming to generate synthetic tumor data mirroring tumor clonal evolution. This is done by introducing mutations in a healthy phased donor genome (DNA) by subsampling reads for every proposed tumor clone. An embodiment of the present builds upon the pipeline to provide additional improvements, which are discussed above. Moreover, improvements over BAMSurgeon as further developed and extended by Salcedo et al. also include, for example: (i) OncoCloneSim includes splicing variants and gene fusions to further improve accuracy, and (ii) instead of using synthetic mutations, OncoCloneSim samples tumor-specific DNA mutations and RNA variants directly from a pool of tumor samples, which offers the advantage of analyzing actual neoantigens that are observed in real tumor samples, thereby providing more realistic and relevant data for further analysis.

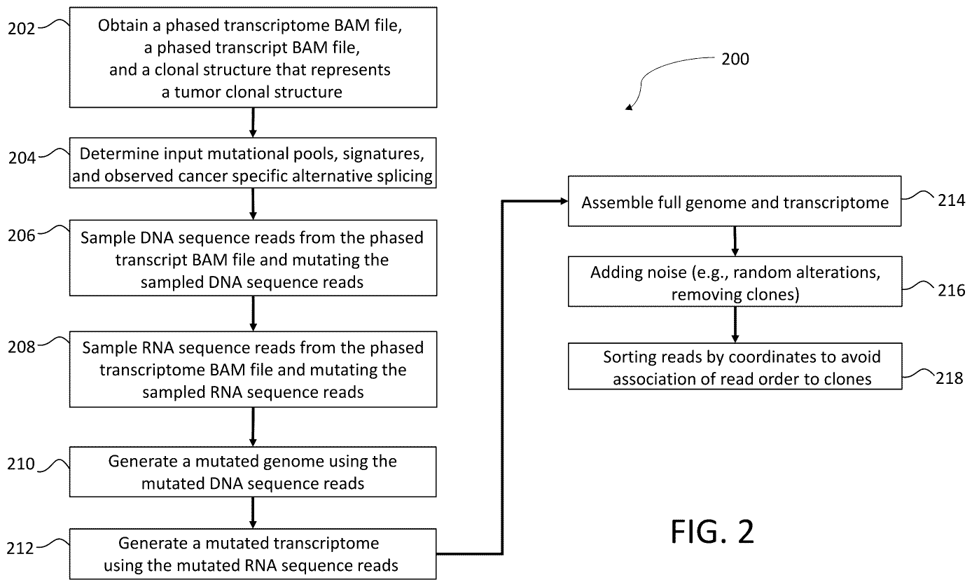

FIG. 2 is a flow diagram 200 of a method to generate tumor data according to an embodiment of the present invention. Flow diagram 200 includes obtaining a phased transcriptome BAM file, a phased transcript BAM file, and a clonal structure that represents a tumor clonal structure at 202. In embodiments, the clonal structure represents a tumor clonal structure and comprises one or more nodes. The input for embodiments disclosed herein may include matched healthy DNA and RNA aligned BAM files and the desired clonal structure. The healthy DNA and RNA aligned BAM files may be phased. Flow diagram 200 includes determining input mutational pools at 204. The input mutational pools are determined for mutating the phased transcriptome BAM file and the phased transcript BAM file. In embodiments, signatures and observed cancer specific alternative splicing may be determined for mutating the phased transcriptome BAM file and the phased transcript BAM file. The flow diagram 200 includes sampling DNA sequence reads from the phased transcript BAM file and mutating the sampled DNA sequence reads at 206. In embodiments, sampling the DNA sequence reads and mutating the sampled DNA sequence reads includes keeping the mutations of previous parental clones.

The flow diagram 200 includes sampling RNA sequence reads from the phased transcriptome BAM file and mutating the sampled RNA sequence reads at 208. In embodiments, sampling RNA sequence reads and mutating the sampled RNA sequence reads includes adding DNA variants to the transcripts. Flow diagram 200 includes generating a mutated genome using the mutated DNA sequence reads at 210. The flow diagram 200 includes generating a mutated transcriptome using the mutated RNA sequence reads at 212. Flow diagram 200 includes assembling a full genome and transcriptome at 214. The flow diagram 200 includes adding noise such as random alterations and/or removing clones at 216. Flow diagram 200 includes sorting reads by coordinates to avoid association of read order to clones at 218.

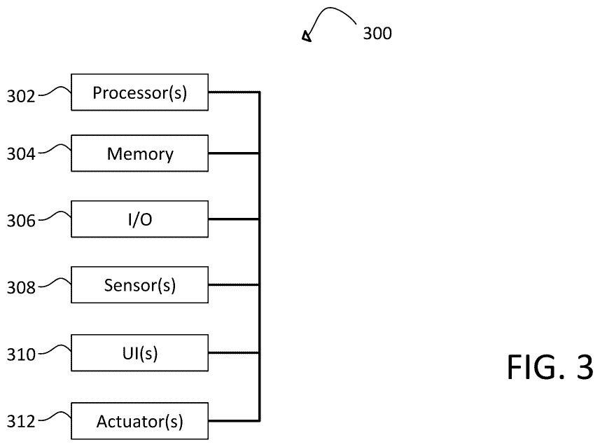

Referring to FIG. 3, a processing system 300 can include one or more processors 302, memory 304, one or more input/output devices 306, one or more sensors 308, one or more user interfaces 310, and one or more actuators 312. Processing system 300 can be representative of each computing system disclosed herein.

Processors 302 can include one or more distinct processors, each having one or more cores. Each of the distinct processors can have the same or different structure. Processors 302 can include one or more central processing units (CPUs), one or more graphics processing units (GPUs), circuitry (e.g., application specific integrated circuits (ASICs)), digital signal processors (DSPs), and the like. Processors 302 can be mounted to a common substrate or to multiple different substrates.

Processors 302 are configured to perform a certain function, method, or operation (e.g., are configured to provide for performance of a function, method, or operation) at least when one of the one or more of the distinct processors is capable of performing operations embodying the function, method, or operation. Processors 302 can perform operations embodying the function, method, or operation by, for example, executing code (e.g., interpreting scripts) stored on memory 304 and/or trafficking data through one or more ASICs. Processors 302, and thus processing system 300, can be configured to perform, automatically, any and all functions, methods, and operations disclosed herein. Therefore, processing system 300 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 300 can be configured to perform task “X”. Processing system 300 is configured to perform a function, method, or operation at least when processors 302 are configured to do the same.

Memory 304 can include volatile memory, non-volatile memory, and any other medium capable of storing data. Each of the volatile memory, non-volatile memory, and any other type of memory can include multiple different memory devices, located at multiple distinct locations and each having a different structure. Memory 304 can include remotely hosted (e.g., cloud) storage.

Examples of memory 304 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 304.

Input-output devices 306 can include any component for trafficking data such as ports, antennas (i.e., transceivers), printed conductive paths, and the like. Input-output devices 306 can enable wired communication via USB®, DisplayPort®, HDMI®, Ethernet, and the like. Input-output devices 306 can enable electronic, optical, magnetic, and holographic, communication with suitable memory 306. Input-output devices 306 can enable wireless communication via WiFi®, Bluetooth®, cellular (e.g., LTE®, CDMA®, GSM®, WiMax®, NFC®), GPS, and the like. Input-output devices 306 can include wired and/or wireless communication pathways.

Sensors 308 can capture physical measurements of environment and report the same to processors 302. User interface 310 can include displays, physical buttons, speakers, microphones, keyboards, and the like. Actuators 312 can enable processors 302 to control mechanical forces.

Processing system 300 can be distributed. For example, some components of processing system 300 can reside in a remote hosted network service (e.g., a cloud computing environment) while other components of processing system 300 can reside in a local computing system. Processing system 300 can have a modular design where certain modules include a plurality of the features/functions shown in FIG. 3. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for generating clone-specific tumor data, the method comprising:
obtaining a phased transcriptome file, a phased transcript file, and a clonal structure that represents a tumor clonal structure and comprises one or more nodes, for each node of the clonal structure:
determining input mutational pools for mutating the phased transcriptome file and the phased transcript file;
sampling Deoxyribonucleic Acid (DNA) sequence reads from the phased transcript file and mutating the sampled sequence DNA reads;
sampling Ribonucleic Acid (RNA) sequence reads from the phased transcriptome file and mutating the sampled sequence RNA reads;
generating a mutated genome using the mutated DNA sequence reads; and
generating a mutated transcriptome using the mutated RNA sequence reads.

2. The computer-implemented method according to claim 1, further comprising modifying the mutated genome or the mutated transcriptome by inserting random alterations.

3. The computer-implemented method according to claim 1, further comprising modifying the mutated genome or the mutated transcriptome by removing one or more nodes of the clonal structure.

4. The computer-implemented method according to claim 1, wherein determining the input mutational pools for mutating the phased transcriptome file and the phased transcript file includes restricting a pool of mutations using observed cancer-type specific mutational signatures associated with the clonal structure.

5. The computer-implemented method according to claim 4, wherein determining the input mutational pools for mutating the phased transcriptome file and the phased transcript file further includes restricting the pool of mutations using observed cancer-type specific alternative splicing and gene fusion events, and wherein determining the input mutational pools and restricting the pool of mutations includes updates from databases.

6. The computer-implemented method according to claim 1, wherein the phased transcriptome file and the phased transcript file are Binary Alignment/Map (BAM) files.

7. The computer-implemented method according to claim 1, wherein generating the mutated genome using the mutated DNA sequence reads includes merging sequence reads simulated from the mutated DNA sequence reads of each node of the clonal structure.

8. The computer-implemented method according to claim 7, wherein merging the sequence reads simulated from the mutated DNA sequence reads further includes using parameters identifying sequencing error rates and down sampling from each node of the clonal structure.

9. The method according to claim 1, wherein sampling the RNA sequence reads includes using a negative binomial distribution.

10. The method according to claim 1, wherein mutating the sampled RNA sequence reads includes adding DNA variants.

11. The method according to claim 10, wherein mutating the sampled RNA sequence reads includes augmenting with genomic mutations specific to a node of the clonal structure that corresponds to the DNA at the same position as the RNA for the node.

12. The method according to claim 1, further comprising sorting sequence reads by coordinates of the mutated DNA sequence reads and the mutated RNA sequence reads.

13. The computer-implemented method according to claim 1, wherein sampling the DNA sequence reads and the RNA sequence reads includes using sliding window sampling.

14. : A computer system comprising one or more hardware processors which, alone or in combination, are configured to provide for execution of a method for generating tumor data comprising the following steps:
obtaining a phased transcriptome file, a phased transcript file, and a clonal structure that represents a tumor clonal structure and comprises one or more nodes, for each node of the clonal structure:
determining input mutational pools for mutating the phased transcriptome file and the phased transcript file;
sampling Deoxyribonucleic Acid (DNA) sequence reads from the phased transcript file and mutating the sampled sequence DNA reads;
sampling Ribonucleic Acid (RNA) sequence reads from the phased transcriptome file and mutating the sampled sequence RNA reads;
generating a mutated genome using the mutated DNA sequence reads; and
generating a mutated transcriptome using the mutated RNA sequence reads.

15. A tangible, non-transitory computer-readable medium having instructions thereon, which, upon being executed by one or more processors provide for execution of a method for generating tumor data comprising the following steps:
obtaining a phased transcriptome file, a phased transcript file, and a clonal structure that represents a tumor clonal structure and comprises one or more nodes, for each node of the clonal structure:
determining input mutational pools for mutating the phased transcriptome file and the phased transcript file;
sampling Deoxyribonucleic Acid (DNA) sequence reads from the phased transcript file and mutating the sampled sequence DNA reads;
sampling Ribonucleic Acid (RNA) sequence reads from the phased transcriptome file and mutating the sampled sequence RNA reads;
generating a mutated genome using the mutated DNA sequence reads; and
generating a mutated transcriptome using the mutated RNA sequence reads.

16. The computer-implemented method according to claim 1, wherein the generated tumor data is used to support decision making in medical or healthcare domains.
